(19) United States
(12) Patent Application Publication
(10) Pub. No.: US 2008/0206751 A1
(43) Pub. Date: Aug. 28, 2008

(54) Method For Carrying Out A Multi-Step Reaction, Breakable Container For Storing Reagents And Method For Transferring Solid Reagent Using An Electrostatically Charged Wand

(75) Inventors: David James Squirrell, Wiltshire (GB); Martin Alan Lee, Wiltshire (GB)

Correspondence Address: POLSINELLI SHALTON FLANIGAN SUELTHAUS PC, 100 S. Fourth Street, Suite 100, St. Louis, MO, 63102, US

(73) Assignee: ENIGMA DIAGNOSTICS LTD, Wiltshire, GB (GB)

(21) Appl. No.: 11/814,726

(22) PCT Filed: Jan. 26, 2006
(86) PCT No.: PCT/GB06/00264

§ 371 Date: Sep. 27, 2007

(30) Foreign Application Priority Data
Jan. 26, 2005 (GB) 0501623.3
Feb. 19, 2005 (GB) 0503481.4

(51) Int. Cl. C12Q 1/68; B01L 3/00; C12P 19/34; B65G 61/00; B65H 29/00

(52) U.S. Cl. 435/6; 435/91.2; 422/102; 414/801; 414/788

(57) ABSTRACT

The application relates to a method of performing a multi-step reaction vessel (68) having at least two compartments (685, 680). The reagents are placed in the first compartment (685) and moved to second one (680) by centrifugation, after which another set of reagents may be placed in the first compartment (685) while the reaction in the lower chamber takes place. Once the reaction is complete, the reagents that were in the first compartment (685) may be moved to the lower one (680) by centrifugation. The application also claims a container having a pierceable lower surface and an upper surface with either a pierceable component or a lid. A wand capable of being electrostatically charged, an apparatus comprising such a wand and a method of transferring solid reagents using such a wand is also claimed.

This invention relates to a method for carrying out a multi-step reaction such as a chemical or biochemical reaction, and in particular an amplification reaction such as the polymerase chain reaction, in which a subsequent step such as analyte detection or further amplifications are effected, as well as elements such as reagent containers and reagent transfer means which may be used in these methods.

There are very many instances where chemical or biochemical assays or reactions are carried out in multiple reaction steps, in the sense that a first reaction is carried out, and after this, one or more further reagents are required to be added to carry out a second reaction, or to provide an indicator that the first reaction has proceeded. The introduction of the one or more further reagents can give rise to contamination problems, in particular where it is necessary to remove a cap or lid from the reaction vessel after the first reaction to allow for the addition of the one or more further reagents.

Amplification reactions are particularly prone to carry-over contamination, because of the very low quantities of starting reagent required. Even minute traces of products such as previously amplified products may contaminate and thereby “seed” further reactions.

Problems are exacerbated where it is required that the reaction is carried out automatically, since removal of caps and the like is not generally easy to achieve in an automated device. As a result, these may be conducted in open reaction vessels, and so the risk of contamination remains.

More recently, a number of closed tube assays have been developed. With these assays however, it is necessary that the amplification and detection reagents are in a homogenous system. Although these are now available, there are sometimes reasons where non-homogenous methods may be preferred, in particular where detection agents required to be used may, to a greater or lesser extent, inhibit the amplification reaction.

The applicants have found an improved way of conducting multi-step reactions.

According to a first aspect of the present invention there is provided a method for carrying out a multi-step reaction, said method comprising

1)adding one or more first reagents to a reaction vessel, said reaction vessel comprising an upper chamber capable of holding reagents, which is open to a lower chamber to which reagent flow is restricted,

2) subjecting said reaction vessel to a centrifugal force so as to drive the said one or more first reagents into the lower chamber;

3) adding a further reagent to the first chamber and closing said chamber;

4) subjecting at least one of the lower chamber or the upper chamber to conditions which cause said one or more first reagents or said further reagent respectively to take part in a first reaction or reach a desired reaction condition;

5) subjecting said reaction vessel to a centrifugal force so as to drive the said further reagent into the lower chamber and allowing it to interact with contents of the lower chamber;

wherein at least steps (2) to (5) are carried out automatically.

By closing the reaction vessel after addition of the further reagent, the possibility of subsequent outside contamination occurring, for instance whilst the first reaction is carried out or the desired reaction condition is reached, is effectively eliminated.

Suitably the lower chamber comprises a restricted access tube such as a capillary or other small tube, into which the reagents will not, under normal circumstances, flow, for instance as a result of surface tension. The tube will be closed at its lower end.

For reactions in which a material is to be heated or cooled it is preferred that the chamber has a high surface area to volume ratio, so that rapid heat exchange can occur, and a capillary tube provides a good example of such a chamber. These tubes are capable of being used in the rapid heating or cooling of small volumes of fluid samples.

Thus in a particularly preferred embodiment, during step (4) it is the lower chamber which is subjected to the requisite conditions.

The reaction vessel is suitably closed during step (3) above by means of an appropriately shaped lid, which can be snap fitted or screwed into place over the mouth of the reaction vessel to form an airtight seal. However, other closure methods and means, for example, using sealant films, metal foils or laminated metal membranes, which are applied over the mouth of the reaction vessel may also be used. Furthermore, as illustrated hereinafter, in certain apparatus, particularly automated apparatus, where samples and the like are delivered automatically into the reaction vessel, other components, such as delivery nozzles, delivery wands (for instance where transfer of materials is achieved through the use of magnetic beads and magnetic rods), or cutters or piercing wands can be adapted to act as a means for also closing the reaction vessel.

The one or more first reagents, and the further reagent may be a combination of reagents which react together only when subjected to particular conditions, such as heating and/or cooling or irradiation for instance with U.V. light, which can be applied during step (4) above. Alternatively, the one or more first reagents may be intended to react in a preliminary step with one or more additional reagents, which have already been dispensed into the lower chamber, for instance, in a preliminary centrifugation step. If appropriate, any pre-dispensed reagents may be freeze-dried within the lower chamber.

However, the reagents do not have to take part in a reaction during step (4). It may be desirable simply to ensure that the one or more first reagents, or the further reagent are brought to a desired reaction condition, for example, to a desired temperature, and optionally held at this temperature for a suitable time, before being mixed together. In this instance, the desired conditions for the desired period of time can be applied during step (4).

The method is widely applicable to a range of reactions. For instance, it may be used in a polymerase chain reaction (PCR), which is interrogated at its end-point. In such a case, the one or more first reagents or the further reagent, but preferably the one or more first reagents comprise a sample containing or suspected of containing a target nucleic acid such as a DNA, as well as the reagents such as primers, buffers, magnesium salts, and polymerase necessary for carrying out a PCR.

If desired, some or all of the reagents necessary for carrying out a PCR, in particular the buffers, polymerase, salts and some stabilisers etc. can be contained in a solid bead, which is added to the upper reaction chamber, and which dissolves or dispenses to release these components on addition of a liquid sample to the upper reaction chamber. Examples of such beads are available commercially for example from Amersham BioSciences UK.

Alternatively, these PCR reagents can be pre-dispensed in the lower chamber, in freeze-dried form, or spun down in a preliminary centrifugation step as described above.

During step 4, the PCR reagents, which are preferably at this stage in the lower chamber, are subjected to the thermal cycling steps necessary to conduct a PCR reaction. This can be done by introducing at least the lower chamber of a reaction vessel into a thermal cycler such a solid block heaters which are heated and cooled by various methods. Current solid block heaters are heated by electrical elements or thermoelectric devices inter alia. Other reaction vessels may be heated by halogen bulb/turbulent air arrangements. The vessels may be cooled by thermoelectric devices, compressor refrigerator technologies, forced air or cooling fluids.

Preferably however, the lower chamber and/or the upper chamber is contiguous with or comprises an electrically conducting polymer, which can itself be utilised as a resistance heater, to effect the heating an cooling. Examples of reactions vessels of this type are described in WO 98/24548.

In particular, the lower chamber will comprise a closed glass tube which is coated around at least the side walls with an electrically conducting polymer. Electrical contacts may be provided at the upper and lower ends of the lower chamber which can be connected to an electrical supply by way of a control device such as a computer, which can be programmed to cause the lower chamber to be sequentially heated and cooled in the manner required in order to carry out a PCR reaction. The upper contact acts as a heat sink, ensuring that any heating regime conducted in the lower chamber will not unduly heat up any further reagent, stored in the upper chamber during this procedure. This may be particularly important if the further reagent is heat sensitive, for example is a reagent used to produce a bioluminescent or chemiluminescent signal.

Particular examples of such reagents comprise a signalling system, which detects DNA and preferably specifically amplified DNA in the sample remaining after an amplification reaction. Such signalling systems may be based upon a variety of properties, but in particular will produce visible signals, which are either fluorescent, chemiluminescent or bioluminescent.

This method can be particularly useful to add a fluorogenic probe that may otherwise inhibit the amplification reaction. For instance, some DNA binding agents, or a high concentration of probes, as well as probes made using DNA analogues such as peptide nucleic acids (PNA) that can, under some circumstances, clamp PCR amplification by inhibition of the polymerase, or by forming extremely stable complexes.

If necessary, the lower chamber may be subjected to conditions such as temperature conditions, which the said signalling requires to be effective. For instance, probes may require that the reaction mixture is heated to denature the DNA present, and then cooled to the temperature at which the probe anneals to the target sequence, which will generally comprise the amplified sequence.

The signalling system is preferably one that can be detected homogenously, without opening the reaction vessel.

Such signalling systems may comprise for example a visible signalling reagent such as a DNA binding agent that emits a different and distinguishable visible-signal when bound to double stranded DNA as compared to when it is free in solution. Examples of such dyes are well known and include ethidium bromide, as well as reagents sold under the trade names of SYBR such as SYBRGreen I or SYBRGold, or other dyes such as YOPRO-1. The presence of significant or high quantities of DNA as indicated by the signal from such a reagent could be indicative that the amplification reaction has proceeded.

Alternatively or additionally, the signalling system may include a labelled probe, which binds specifically to the amplified product. Labels are suitably fluorescent labels, which are detectable following irradiation with light of a suitable wavelength, followed by detection of the resultant emissions from the label. A wide range of fluorescent labels are available commercially. Examples are rhodamine dyes, fluorescein, or cyanine dyes. Particular examples of dyes are sold as Cy5, Cy5.5, TAMRA, ROX, FAM, HEX, TET and JOE.

In a particularly preferred embodiment, the signalling system comprises a combination of a fluorescently labelled probe and a DNA binding agent, which is able to interact with the fluorescently labelled probe, by absorbing fluorescent energy emitted from the probe, or by donating fluorescent energy to the probe. This well-known phenomenon is known are Fluorescence Energy Transfer (FET), or Fluorescent Resonant Energy Transfer (FRET). The donor molecule is excited with a specific wavelength of light which falls within its excitation spectrum and subsequently it will emit light within its fluorescence emission wavelength. The acceptor molecule is excited at the donor emission wavelengths and so accepts energy from the donor molecule by a variety of distance-dependent energy transfer mechanisms. The basis of fluorescence energy transfer detection is to monitor the changes at donor and acceptor emission wavelengths.

In this embodiment, this combination of reagents is added as the further reagent, and the lower chamber is heated and cooled to ensure that the fluorescently labelled probe anneals to the target in the sample where present. The DNA binding agent will intercalate between the duplex formed by the probe and the target and so will interact with the fluorescent label, either by donating fluorescent energy to the label to increase its signal (thereby acting as a fluorescence donor whilst the label on the probe acts as a fluorescence acceptor or quencher), or by quenching the fluorescent signal from the fluorescent label.

The DNA binding agent may be one which is itself fluorescent under these conditions. Preferably however, it is a reagent which is not itself fluorescent, or emits visible light under these conditions, but merely acts as a quencher for the fluorescent label on the probe. In this way, the need for resolving complex visible signals is avoided. Particular examples of compounds which may operate in this way include anthroquinone compounds for instance, DNA binding compounds of formula (I)

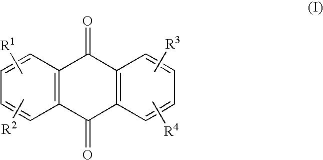

wherein R¹, R², R³ and R⁴ are independently selected from hydrogen, X, NH-ANHR and NH-A-N(O)R′R″ where X is hydroxy, halo, amino, C₁₋₄alkoxy or C₂₋₈alkanoyloxy, A is a C₂₋₄alkylene group with a chain length between NH and NHR or N(O)R′R″ of at least 2 carbon atoms and R, R′ and R″ are each independently selected from C₁₋₄alkyl and C₂₋₄hydroxyalkyl and C₂₋₄dihydroxyalkyl, provided that a carbon atom attached to a nitrogen atom does not carry a hydroxy group and that no carbon atom is substituted by two hydroxy groups; or R′ and R″ together are a C₂₋₆alkylene group which, with the nitrogen atom to which R′ and R″ are attached for a heterocyclic ring having 3 to 7 atoms, with the proviso that at least one of R¹, R², R³ and R⁴ is a group NH-A-N(O)R′R″.

A specific examples of such DNA duplex binding agent is mitoxantrone (

1,4-dihydroxy 5,8-bis[[2-[(2-hydroxyethyl)amino]ethyl]amino]-9,10-anthracenedione) or it salt such as the hydrochloride or dihydrochloride salt.

Other examples of DNA binding agents which do not emit visible signals under these conditions include nogalamycin (2R-(2α,3β,4α,5β,6α,11β,13α,14α)]-11-[6-deoxy-3-C-methyl-2,3,4-tri-O-methyl-α-L-mannopyranosyl)oxy]-4-(dimethylamino)-3,4,5,6,9,11,12,13,14,16-decahydro-3,5,8,10,13-pentahydroxy-6,13-dimethyl-9,16-dioxo-2,6-epoxy-2H-naphthaceno[1,2-b]oxocin-14-carboxylic acid methyl ester) or daunomycin (8S,-cis)-8-acetyl-10-[3-amino-2,3,6-trideoxy-α-L-lyxo-hexopyranosyl)oxy]-7,8,9,10-tetrahydro-6,8,11-trihydroxy-1-methoxy-5,12-naphthacendione).

Suitable combinations of DNA binding agent and fluorescent label for the probe will be understood by the skilled person, or may be determined using routine procedures.

However, chemiluminescent or bioluminescent signalling systems may also be used.

A particular example of a bioluminescent signalling system comprises the luciferase/luciferin system, in which the enzyme luciferase, acts on a substrate luciferin in the presence of ATP to generate light. Luciferase however, is a highly thermosensitive enzyme, and therefore it may not withstand the temperatures which are likely to be produced for instance during a PCR. By using the method of the present invention, such agents can be retained in the upper chamber whilst a reaction at elevated temperature is conducted in the lower chamber, and the signalling system added only when convenient, at the end of the reaction, when the temperature in the lower chamber is reduced to a level at which the luciferase remains active. A particular type of assay which utilises bioluminescent signalling systems in the context of a PCR is described in WO 02/090586, the content of which is incorporated herein by reference. Such a reaction may be particularly amenable to automatic operation using the method described herein.

The signals generated are read using any convenient detection device, for example an optical system such as a spectrofluorimeter, or a camera in the case of chemiluminescent or bioluminescent signalling systems. The device may be included in the apparatus. In the case of an optical detection system, it is preferred that the optical detector is light sealed to ensure that the detection can proceed without interference from non-incident light.

Preferably, the reaction vessel is arranged so that the signal can be read directly from the vessel, for instance through a transparent bottom of the lower chamber, or through a transparent cap or sealing member on the top of the vessel. If necessary however, fluorescent light may be conveyed through a fibre optic, from the reaction vessel

However, there are many other applications of the process, and further reaction stages may be conducted within the lower chamber after step (5) if required. An example of such a process would be a nested or multiplex PCR reaction. In this instance, the further reagent may comprise a further PCR reaction mixture, including different primers, and if necessary further buffers, enzymes etc., in order to conduct a second PCR reaction. In such a case, after the further reagent has been dispensed, the lower chamber is subjected to a further thermal cycling in order to effect the desired second PCR reaction.

This is advantageous for exquisite sensitivity of DNA amplification. It is useful where inhibitors may be present in the sample material. It improves the serious carry-over issues associated with nested PCR for practical application.

The method may also be employed in relation to reverse transcription, (RT-PCR) in various ways. For instance, the one or more first reagents may comprise the reagents necessary for carrying out a reverse transcriptase process, for producing a cDNA from an RNA. These are therefore spun down during step (2), and the PCR reagents required to amplify the cDNA corresponding to the RNA sequence of interest are then added as the further reagent. The lower chamber containing the reverse transcriptase reaction mixture is incubated during step (4) so as to produce the cDNA complementary to the RNA.

The PCR reagents including primers specific for this cDNA are spun down during step (5) above, and an amplification reaction can then be carried out by subjecting the lower chamber to the necessary thermal cycling conditions.

Alternatively, the PCR reagents may comprise the one or more first reagents, which are spun down into the lower chamber during step (2). The reverse transciptase reaction mixture is the further reagent, which is held in the upper chamber. During step (4), the upper chamber is incubated so as to allow the RT reaction to proceed so that cDNA is generated. [For this purpose, it is desirable that the upper chamber is independently heatable/coolable using for instance an electrically conducting polymer as described above]. The thus formed mixture, containing the CDNA is then spun down into the lower chamber during step (5). Thereafter the lower chamber can be subjected to the thermal cycling necessary to allow the PCR reaction to proceed.

The method may also be employed in carry-over prevention measures. For instance, where the first reagents are PCR reagents able to carry out a homogenous detection reaction, and PCR is carried out during step 4, the further reagent, may comprise a reagent able to degrade the products after the contents have been analysed or read. Alternatively, where the detection is conducted using a separate signalling system, added as the further reagent (as described above), the reagent able to degrade the product may by added in a further subsequent step for example by means of a multicompartment cartridge or container, as discussed below.

The compartment containing the degradation agent can then be pierced, and the contents spun down in a subsequent stage of the reaction.

Such reagents may include uracil-n-glycosylase, which is able to degrade dUTP PCR products, or alternatively a suitable DNAse. The latter will not only reduce the risk of carry over, it may also destroy potentially harmful target nucleic acids that may have been within the sample e.g. HBV DNA which is known to be infectious.

The method may also be adapted to produce a form of “HotStart” amplification reaction. Amplification reactions such as PCR reactions rely on the sequence of steps (denaturation, annealing and extension) occurring in a very precise order and at the precise temperature required for the operation of that step. A problem arises when reagents are mixed together, even for short periods of time, at different temperatures, for example prior to the start of the reaction. Primers may interact with nucleic acid template, resulting in primer extension of the template. This can lead to a reduction in the overall yield of the desired product as well as the production of non-specific products.

Various means of overcoming this problem have been proposed previously, and these have become known as “Hot Start” reactions.

By using a method in accordance with the invention, it would be possible to retain one or more of the reagents necessary for carrying out the amplification reaction, for instance magnesium ions, the polymerase or the primers, as the further reagent. This may then be dispensed only when the conditions within the lower chamber are favourable to the correct amplification occurring, for instance, it has been heated to a temperature in excess of that at which small DNA molecules associate.

Any heating or cooling of the upper or lower chambers of the reaction vessel is suitably automated, for instance using a computer to control the supply of current to the thermal cycler. The computer is suitably programmed to ensure that the desired sequence of temperature steps are achieved.

The one or more first reagents and/or the further reagent may be dispensed into the reaction vessel during steps (1) and (3) respectively using any convenient method. For instance, the reagents may be dispensed into the upper chamber by a conventional injection technique, which is preferably carried out automatically using a suitable dispensing apparatus.

Alternatively, the one or more first reagents and/or the further reagent, may be arranged in a breakable container, such as a cartridge, disposed within the upper chamber above the opening into the lower chamber.

These may, for instance have pierceable walls at the upper and/or lower surface of a reagent chamber, such as metal or laminated metal membrane surfaces. The contents can then be released at the appropriate stage, by breaking the walls of the chamber, for instance by introducing a piercing wand or pin through the pierceable walls, so that the reagent is released through the bottom of the chamber.

The piercing wand or pin may be provided on the cap of the vessel, which suitably forms the upper surface of the reagent chamber, and may be introduced by applying pressure to the cap in the appropriate time. Alternatively, it may be provided on the machine used to conduct the reaction, and applied automatically at the appropriate time, in particular where the upper wall itself is pierceable.

The upper surface of a sealed cartridge or container may comprise for instance a membrane, such as a plastics membrane, and have suitable piercing wands or pins incorporated therein. In this embodiment, the upper surface of the container may itself comprise the cap of the vessel. Alternatively, a separate cap is provided and the wand may be required to pierce this cap before reaching the upper surface of the container, to minimise contamination risk.

Particular containers are in the form of cartridges which have more than one compartment. These may contain the one or more first reagents and/or the further reagent respectively. However, they may also open up the possibility that additional reagents may be added automatically, either at the same time, or sequentially, to the upper chamber at appropriate times during the method, and spun down as required, giving rise to the possibility that any number of further reaction steps can be conducted, and/or reagents which cannot be stored together are not mixed until they reach the reaction chamber.

Such cartridges will further minimise the contamination risk, as they ensure that the reagents are not exposed to the atmosphere for longer than is necessary.

These compartments may be arranged adjacent each other, for instance, in a circular or wheel-like arrangement, or they may be arranged on top of each other. In either case, one or more suitable wands are provided to allow the compartments to be breached as necessary during the method.

The container may be provided at its upper region with means to allow the container to be moved automatically into position within the reaction vessel. Examples of such means may comprise one or more annular flanges, which are arranged to interact with suitable grabber means on the apparatus in which the method is conducted.

By using reagent containers of this type, any combination of the reactions described above, for example nested PCR, RT-PCR, non-homogenous detection, carry-over prevention and/or hotstart PCR may be carried out sequentially, merely by delivering the appropriate reagents in the appropriate sequence and at the appropriate time.

Such containers form a further aspect of the invention. Thus in accordance with a further aspect of the invention, there is provided a breakable container for storing reagents, said container having therein a reagent chamber with at least one pierceable walls at the lower surface thereof, and wherein the upper surface comprises either a further pierceable wall, or a lid comprising a piercing means, arranged such that piercing of said pierceable walls leads to release of reagent from the chamber.

The reaction vessel is suitably mountable on a platform or the like, which can be rotated in a centrifugal motion. The reaction vessel is suitably pivotally mounted on the platform, so that during centrifugation, it is able to turn to cause the lower chamber to be located at the outer centrifuge path. This may be achieved for instance by means of one or more spindles, provided on the outer surface of the vessel, which may be movably mounted in sockets providing on the platform.

It is also moveable automatically, between stages. For instance, it may be appropriate to move the reaction vessel into a thermal cycling means for the processing steps. In the preferred embodiment, where the reaction vessel is provided with heating means consisting of electrically conducting polymer, these are moved using automatic moving equipment from a centrifuge, into an appropriate socket in an electrical supply, which makes contacts with the electrical contacts on the reaction vessel. Alternatively, electrical contacts may be made in-situ.

The provision of automatic means for conducting the method means that the amount of manual handling of the samples is minimised, making the process efficient, and reducing still further the risk of contamination, in particular from operator DNA.

Particularly suitable reaction vessels and apparatus for use in the method described above are set out in co-pending International Patent Application Publication No. WO2005019836, the content of which is incorporated herein by reference. The apparatus described here is able to carry out highly complex multi step processing of samples. The apparatus comprises:
i) a platform comprising:
   (a) a chamber suitable for receiving a sample; and
   (b) a functional component;
(ii) an arm capable of being raised and lowered and including a means for removeably attaching to the functional component such that said component may be raised and lowered with the arm; and
(iii) a means for moving the platform such that any chamber or functional component may be aligned with respect to the arm.

This apparatus has a wide variety of applications, and can be adapted for a wide variety of uses. The term “functional component” is defined as meaning an element of the apparatus that has been designed such that it can attach reversibly to the arm of the apparatus. The functional component can be designed to have a wide variety of uses as will be apparent from the disclosure herein. The specific use of one or more functional components can be readily identified by one skilled in the art depending on the specific use of the apparatus. For example the functional component may comprise a means for interacting with the fluid sample. Such a means may provide some physical processing to the sample for example heating, cooling, optics, sonication, and the like. Alternatively the functional component may comprise a means for interacting with the chamber itself, for example by acting as a cutter to pierce a foil seal, to cap the chamber, to introduce a filter and the like. Furthermore the functional component may act as a collector for moving the sample, or an analyte contained therein to another chamber of the apparatus.

In the context of the present method therefore, the functional component may comprise means for delivering a sample, which may have been pretreated elsewhere in the apparatus to the reaction vessel, which in this case will be the equivalent of the “chamber suitable for receiving a sample”. Alternatively, the functional container may comprise a cutter or piercing wand for releasing reagents contained in cartridges or breakable containers as described above.

In a particular embodiment however, the platform is essentially circular and moves by rotation. This allows the platform to align the chambers or functional components with respect to the arm or other physical means. This also has the advantage of minimising the size of the apparatus when several different components are involved. Optionally the platform can be fitted with a sensing mechanism to allow for correct positioning of the functional component or chamber as the platform moves under the arm or other physical processing means located above the platform. However, in the context of the present method, it is further advantageous in that it allows for the easy centrifugation of a reaction vessel held on the platform.

In a further particular embodiment, the apparatus is designed such that the whole platform can be removed and readily replaced. This allows that after any given sample processing sequence, the used and potentially contaminated platform can be removed and replaced to allow use of the apparatus in a further procedure. If the apparatus is so designed it is preferred that the platform can be readily and securely mounted into the apparatus for easy of use for example using a twist fit with a simple lock.

Automatic handling of the reagents utilised in the method of the invention may be further enhanced by the use of reagent transfer devices. The applicants have further developed methods of handling reagents used in chemical and biochemical assays, as well as devices used in these methods.

There is frequently a need to transfer small quantities of reagents, in solid form, from one vessel to another in the course of conducting chemical or biochemical assays.

Increasingly, these assays are conducted using automated processes and procedures, as required in the method of the invention and in apparatus as such as that described in WO2005019836, and so automated devices capable of picking up and transferring quantities of reagents are required.

Particular examples of reagents, which are available in solid form, are the reagents necessary for carrying out an amplification reaction, in particular the Polymerase Chain Reaction or PCR. Commercial PCR ready-to-go beads are available for carrying out DNA amplification, such as those sold by Amersham BioSciences UK.

They contain all the components necessary to carry out standard PCR including buffer reagents, salts and polymerase enzymes, in a freeze-dried or other solid form. They therefore provide a convenient means of providing stabilised “homebrew” consumables for end user assembly. They benefit from mass production and a reproducible formulation in a convenient storage format that has a long shelf life.

However, unlike wet reagents that are easily transferred between wells using a pipette or pipettor, beads require tweezers to transfer from one consumable to another. This can be difficult to achieve efficiently, in particular in automated devices.

The applicants have developed an efficient method of manipulating solid reagents effectively.

Thus in a further aspect, the present invention provides a method for transferring solid reagents from a first container or first position to a second container or second position, said method comprising
(i) bringing into the vicinity of said solid reagents in the first container or first position a wand comprising an electrostatically charged material, said wand being capable of electrostatically attracting and retaining said solid reagent on the surface thereof, so as to pick up a quantity of said solid reagent,
(ii) moving the wand and/or the first or second containers so that the wand is in the vicinity of the second container or position,
(iii) removing the solid reagent from the said wand, so that it is placed in the second container or second position.

In the context of the first aspect of the invention, the second container or position will comprise the upper chamber of the reaction vessel. However, the method may be more widely applicable, to transfer reagents generally.

The method is extremely useful in that it allows the efficient transfer of solid reagents. Furthermore, it is particularly amenable to automation and may be included in a wide range of assay devices.

The term “solid reagent” as used herein refers to one or more agents or chemicals which are in solid form. For instance, they may comprise powders, crystals, granules or beads. In particular, they comprise a combination of reagents, which are combined together in a bead form, such as the PCR ready-to-go beads, as described above. Where reagents are generally found in a liquid form such as in solution in water, suitable solid forms may be prepared by conventional methods such as freeze-drying or spray drying. The beads or granules may further comprise conventional agents such as fillers, dispersants, surfactants etc. to ensure that the granules or beads dissolve or disperse when added to liquids such as water, if that is required.

The nature of the electrostatically charged material will vary depending upon the nature of the solid reagents being transferred. Typically, the material will comprise a dielectric material that is an insulator or non-conductor, or has negligible electrical conductivity. The material should be one that generally carries or is able to retain for a reasonable period, a static charge. Particular examples of such materials are polymer or plastics material, in particular, polystyrene or latex.

Further, it is possible to increase or to generate sufficient charge on the wand by a preliminary rubbing step, in which the wand is mechanically rubbed one or more times against an insulator such as a material or fabric, so as to generate or increase the static charge. Suitable materials will comprise synthetic fabrics such as nylons or polyester fabrics.

This is suitably carried out automatically, preferably at a suitably arranged operating station within an automatic assay device.

As used herein, the term “wand” refers to any suitable structure which may be introduced into and moved between reaction vessels and the like. Generally it will be elongate in shape, for example a rod-like tubular structure, although the sides may be inclined, so as to form a syringe-like or conical profile.

It may be solid or hollow in nature. Where the wand is hollow, it may be able to accommodate additional elements, such as magnets. In this case, introduction of a magnet into a wand may allow it to be used also for the collection of magnetic solids, such as magnetic beads, like magnetic silica beads, which optionally carry further reagents such as binding reagents like antibodies or binding fragments thereof. Such collection may be carried out in the same operation as the electrostatic retrieval of solid reagents, but is preferably conducted as a separate operation.

The entire wand may be made of a dielectric or electrostatically chargeable material, or it may comprise a composite, provided that the area intended to attract the solid reagent, in particularly a lower surface or region, comprises a dielectric or electrostatically chargeable material.

Suitably, at least a part of the outer surface of the wand is profiled to allow reagents to be accommodated within the profiles. For example, the surface may be provided with dimples or grooves, which are of a suitable size to accommodate the solid reagents such as the amplification reagent bead, for instance PCR beads.

The size of these grooves or dimples will depend upon the size of the solid reagent such as the amplification reaction or PCR bead which is going to be moved using the wand. Generally however, any dimples will be from 0.5-2 mm diameter and depth, and similarly grooves will be from 0.5 to 2 mm wide and deep.

Any profiling is suitably arranged on a lower surface of the wand.

Suitably profiled wands may be novel and these form a further aspect of the invention.

The movement effected in step (ii) above can be carried out in any way suited to the particular assay and assay device being used. After collection of the solid reagent, the wand may be moved manually from one place to another, but the operation is suitably carried out automatically. This may for example involve movement of the wand vertically upwards, for example to remove it completely from the first container, and then horizontally so that it is aligned with the second container, and if necessary downwards so that an end region of wand, on which the solid reagent is retained, is within the second container. Alternatively, after removal of the wand from the first containers by for instance an upwards movement of the wand, the containers themselves may be moved so that the second container is arranged below the wand. The wand may then simply be lowered so that it enters the second container as necessary.

Suitable transport means are well known in the art, and may comprise conveyor belts, carousels or the like.

Suitable containers may be any reaction vessel, including individual reaction vessels or reaction wells in plates or the like, and the transport means will be adapted to move these if necessary.

Removal of the solid reagent from the wand in step (iii) is suitably carried out mechanically. In particular, the wand is immersed into a liquid, which will have the effect of sweeping the solid from the wand. Suitably the liquid is a solvent or solution required for the next stage of the assay. For instance, in the case of a solid reagent comprising a PCR bead, this may be dispensed by submerging the wand into the resuspension buffer i.e. the DNA/RNA extract etc to remove the bead from the wand.

Preferably the wand is disposable after use.

The upper end of the wand may be shaped or adapted to fit into the desired fitments or attachments on an automated assay device.

In this context therefore, the wand used in the method of the present invention may comprise a specific functional component of the device of International Patent Application Publication No. WO2005019836 as described above. In particular, it will comprise a component which is suitable for picking up and moving PCR ready to use beads into a reaction vessel, for use in an automated PCR process.

In a preferred embodiment of the device of WO2005019836, the functional component comprises a sheath which provides an interface between a magnet for attracting magnetic reagent beads, which may have immobilised thereon analytes or reactants, and the beads themselves. Preferably the sheath is located on the platform and is made of a material such that when the magnet is inside the sheath the complex will be attracted to the sheath. In such an embodiment it is preferred that the apparatus comprise a magnet co-located with the arm of the apparatus that can be lowered into the sheath to apply a magnetic field and raised out of the sheath to remove the magnetic field. The sheath is then placed into a chamber of the apparatus comprising the magnetic reagent beads. The magnet is lowered into the sheath and the magnetic reagent beads binds to the sheath. The sheath and magnet are then raised. The platform moves such that a new chamber is aligned, the sheath and magnet are then lowered and the magnet removed. When the magnet is removed the magnetic reagent beads will fall away from the sheath into the second chamber. Small movements of the sheath up and down by the arm will ensure that no magnetic reagent beads remains bound to the sheath and will also act to mix the magnetic reagent beads with any reagents or solutions in the new chamber. Alternatively an analyte can be eluted from the beads by any suitable means.

The magnet and the arm are designed to interact with each other without affecting the operation of the other such that the sheath can be independently raised and lowered with or without the magnet in place.

In a particular embodiment, if the sheath is formed as a wand in accordance with the present invention, it may also be used to transfer solid reagents electrostatically as described herein.

Devices particularly adapted for carrying out the above-described reagent transfer method form a further aspect of the invention.

In particular therefore, the invention further provides apparatus comprising a wand comprising an electrostatically chargeable material as described above, and means for transferring said wand from a first container to a second container.

The invention will now be particularly described by way of example with reference to the accompanying diagrammatic drawings in which:

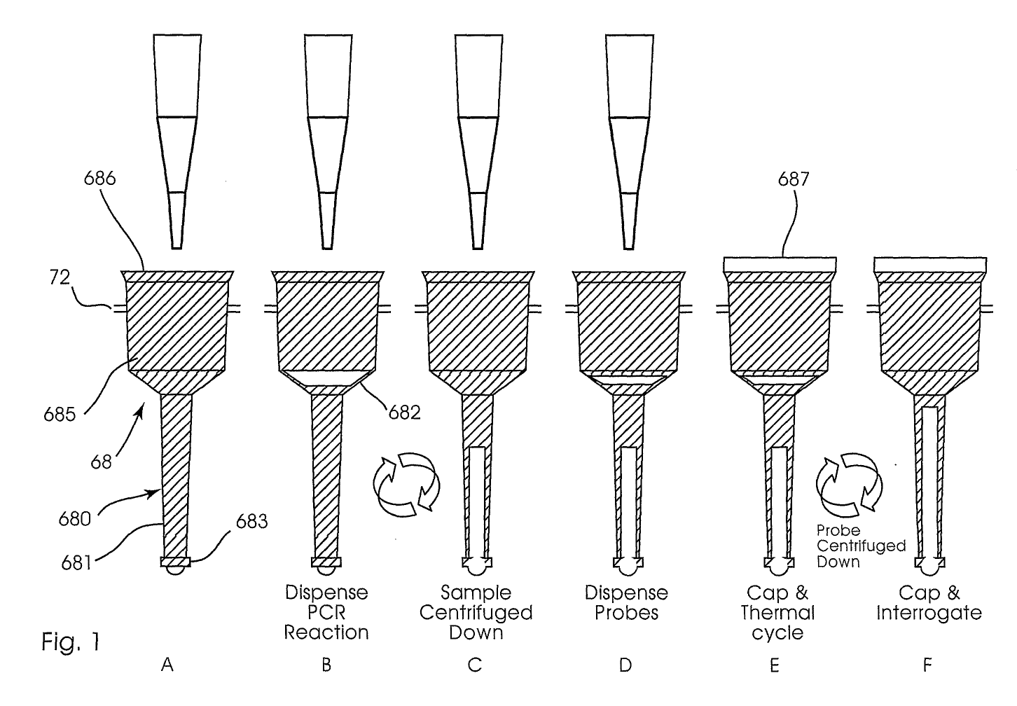

FIG. 1 is a schematic diagram illustrating a method according to the invention;

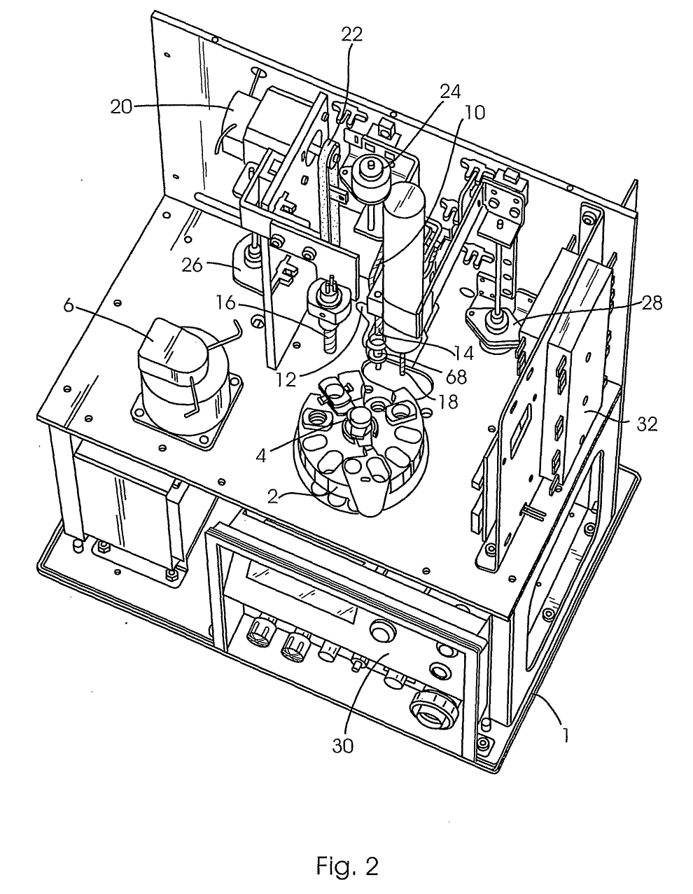

FIG. 2 shows a perspective view of an apparatus which may be used in the method;

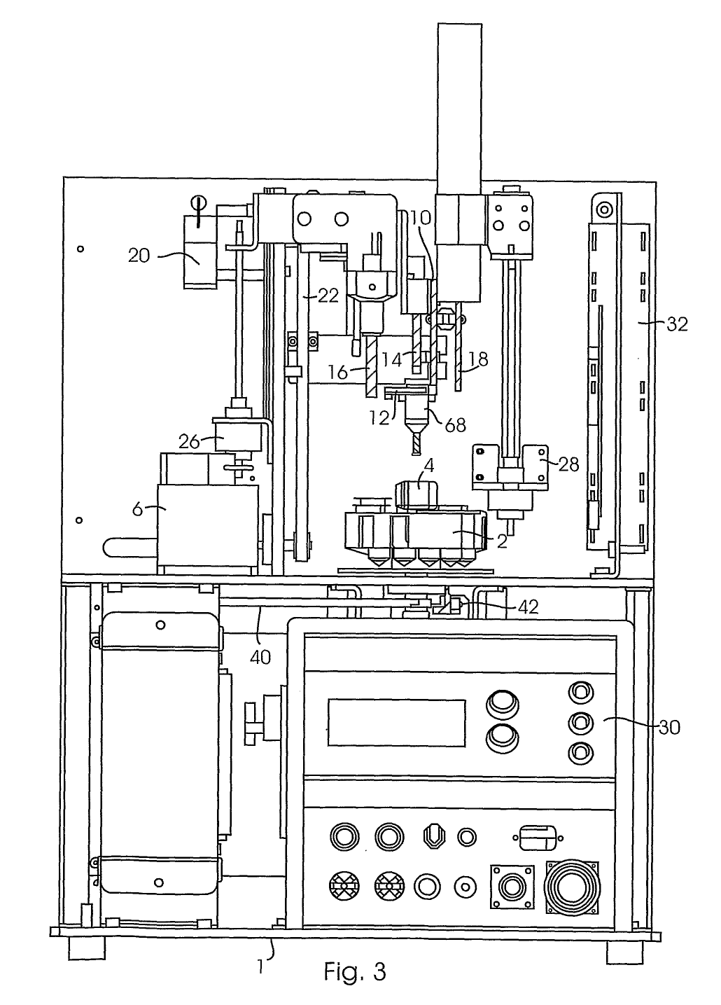

FIG. 3 shows a transverse cross section of the apparatus of FIG. 2 from the side;

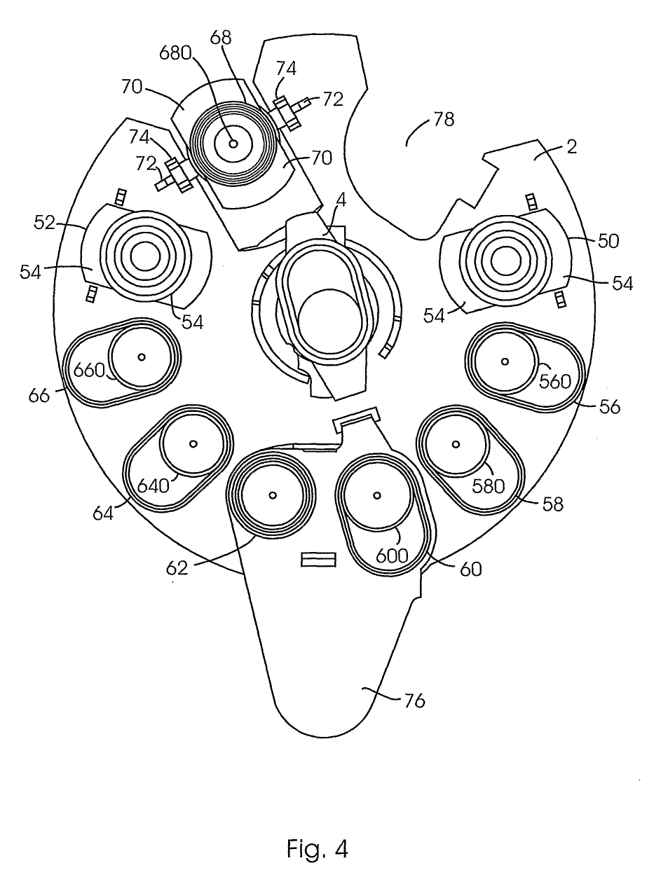

FIG. 4 shows a top view of a platform used in the apparatus of FIGS. 2 and 3;

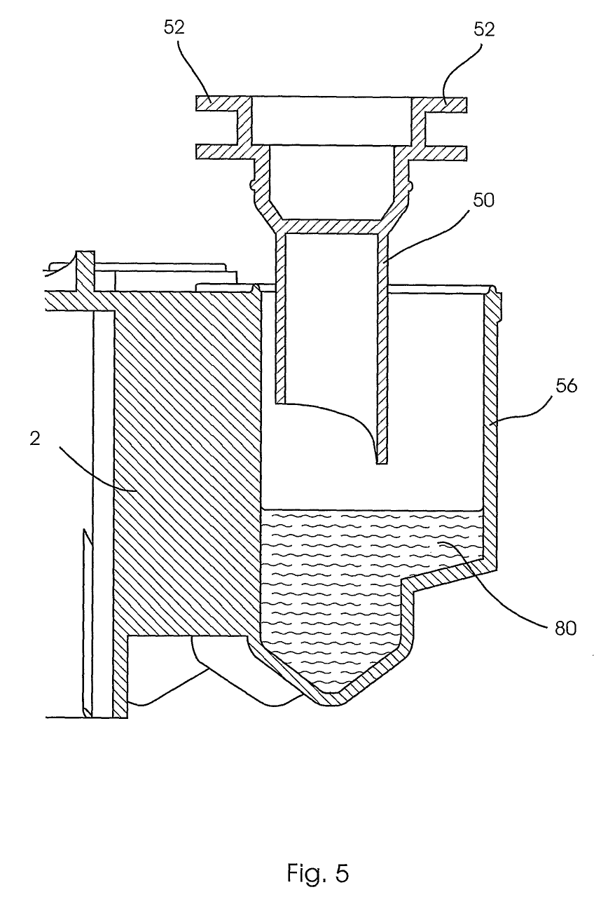

FIG. 5 shows a cross section view of the operation of a functional component, here a cutter, piercing a laminated membrane on a chamber of the apparatus;

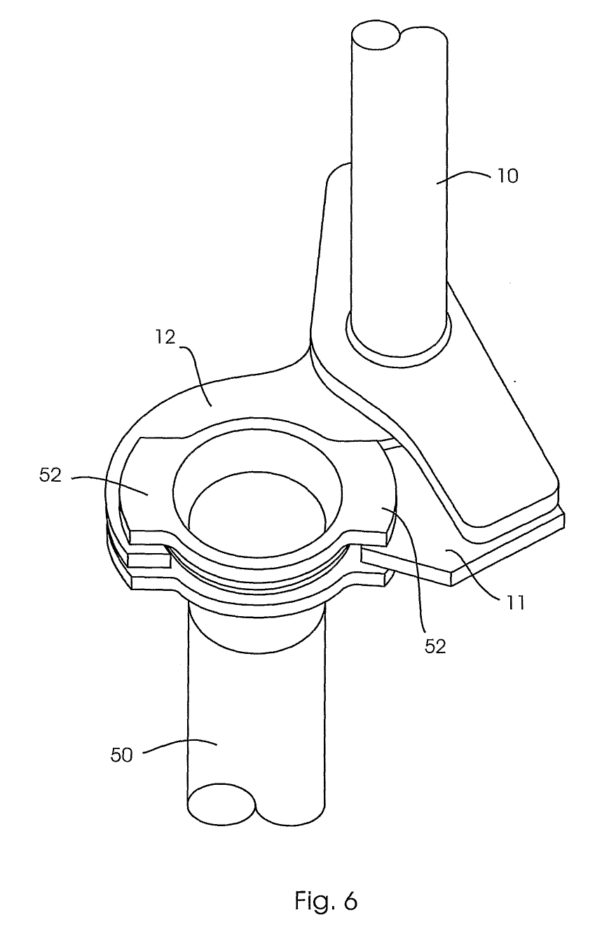

FIG. 6 shows a view to illustrate the detail of the attachment of a functional component, here a cutter, to the fork of the arm;

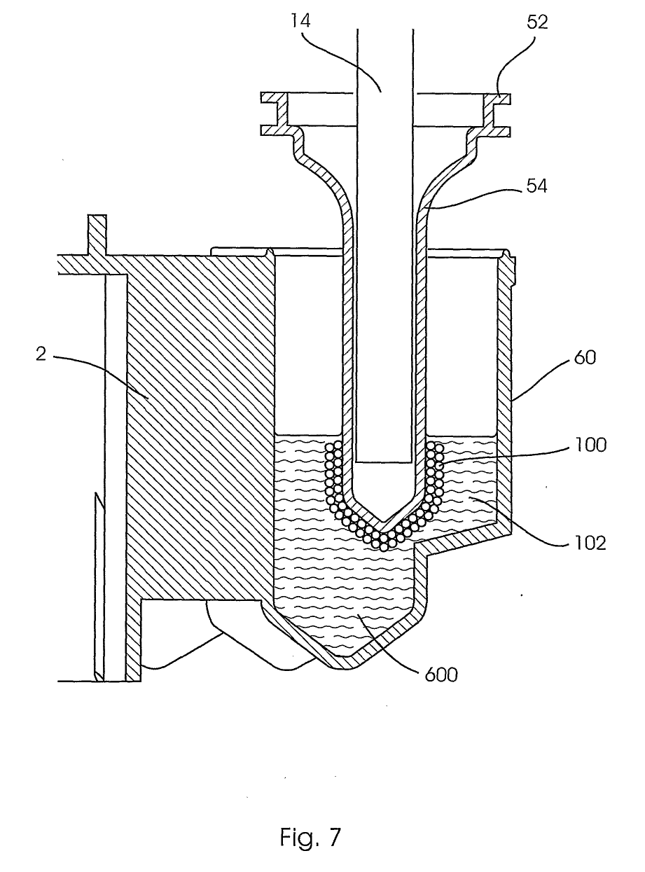

FIG. 7 shows a cross section view of the operation of a functional component, here a sheath, with a magnet to withdraw bound analyte from the sample chamber;

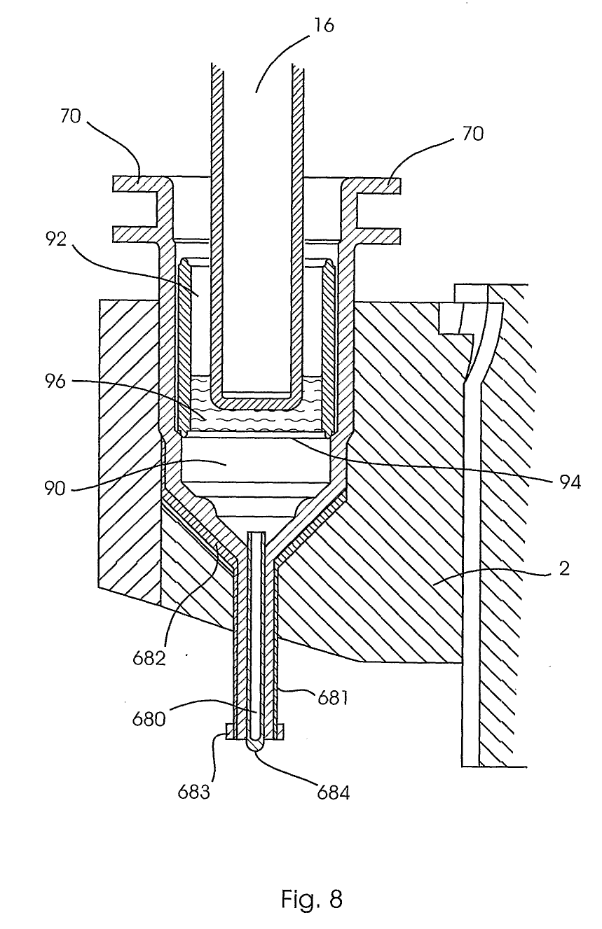

FIG. 8 shows a cross section view of the operation of a physical processing means, here a means for heating, to heat a volume of solution in one of the chambers of the apparatus;

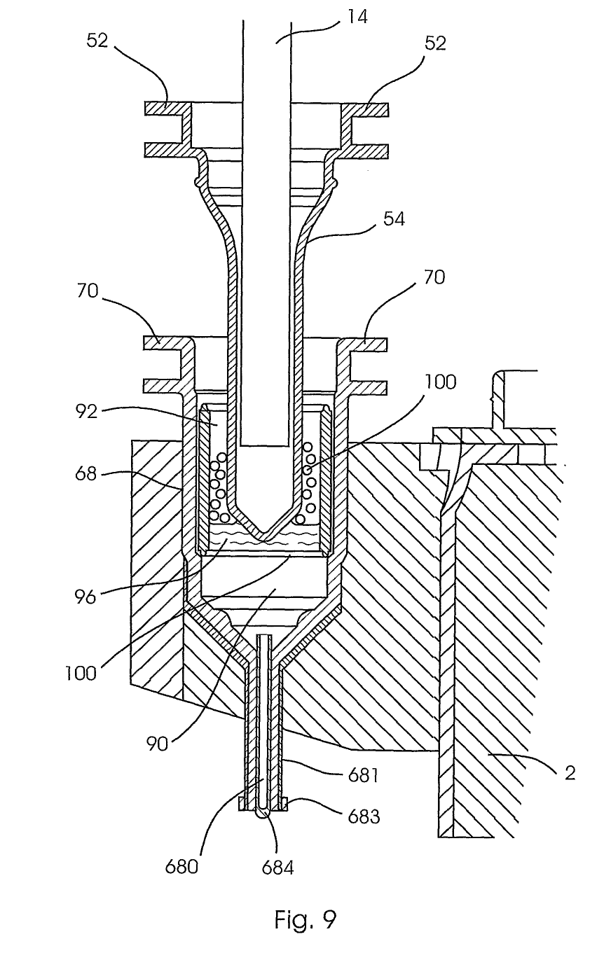

FIG. 9 shows a cross section view of the operation of a functional component, here a sheath, with a magnet to release the bound analyte into the reaction vessel;

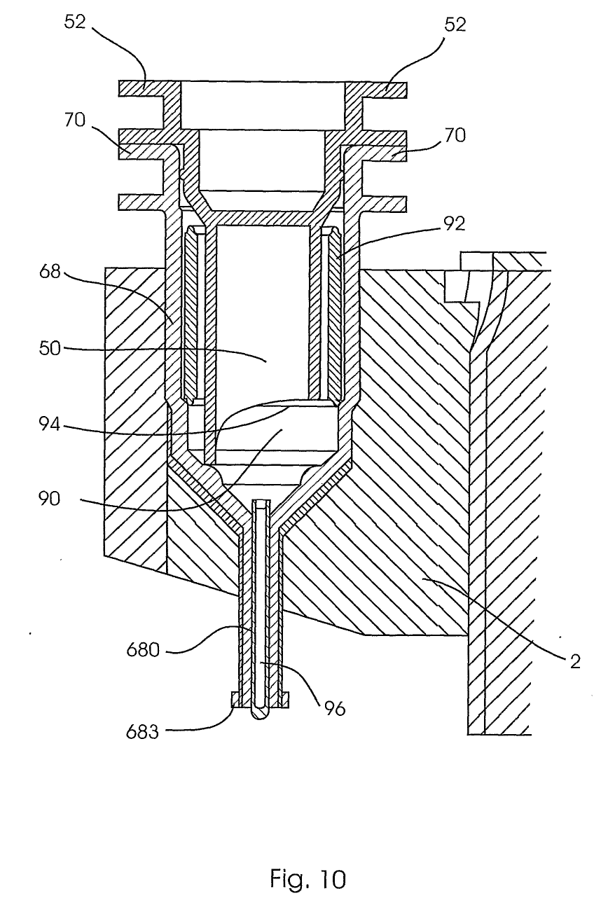

FIG. 10 shows a cross section view of the reaction chamber, here with a functional component, here the cutter, in position to seal the reaction vessel;

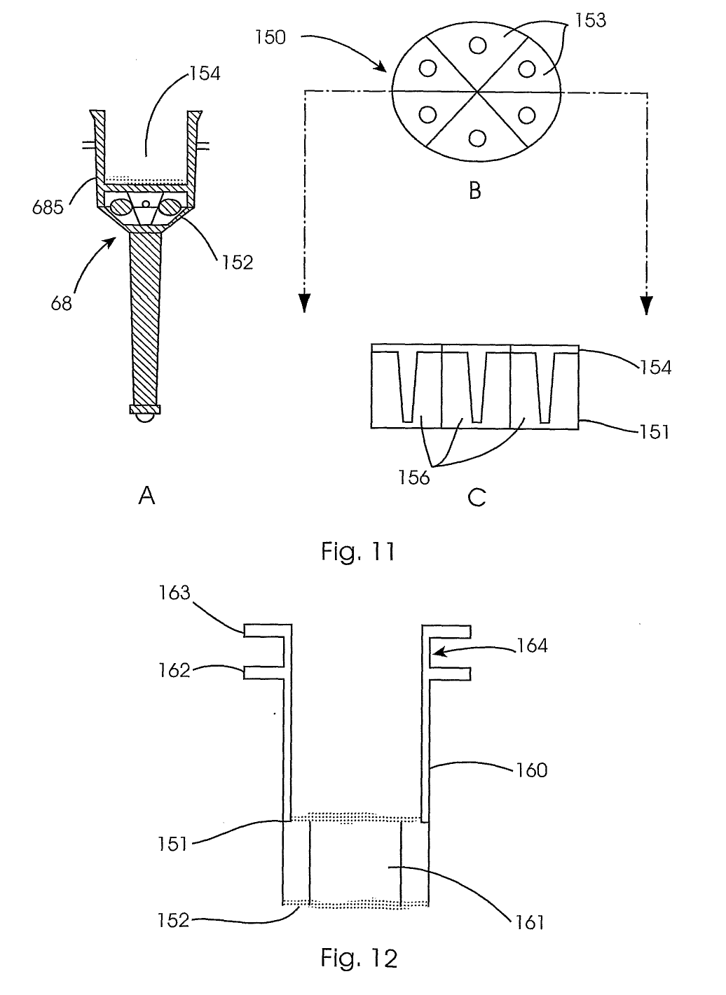

FIG. 11 shows an alternative form of a vessel used in the method of the invention, which allows multiple operations to be conducted, wherein (A) shows a schematic diagram of a reaction vessel with a reagent container in place, (B) is a cross section through the container of (A) and (C) is a schematic side view of the container;

FIG. 12 shows a container which may be used in the method of the invention;

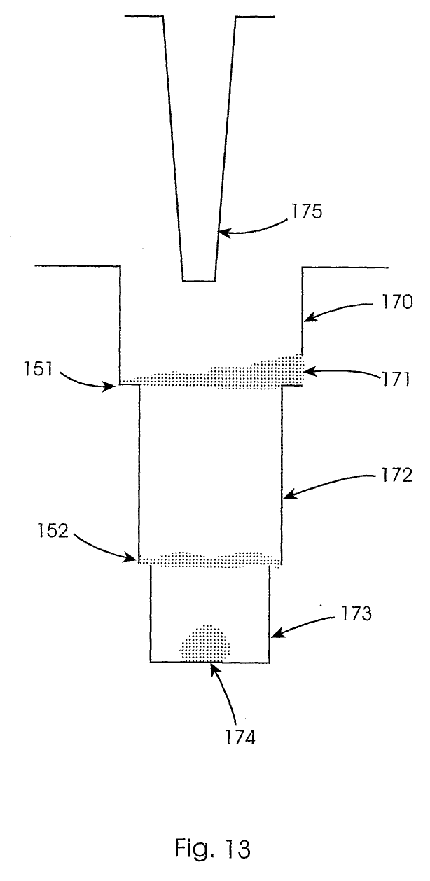

FIG. 13 shows an alternative form of this container; and

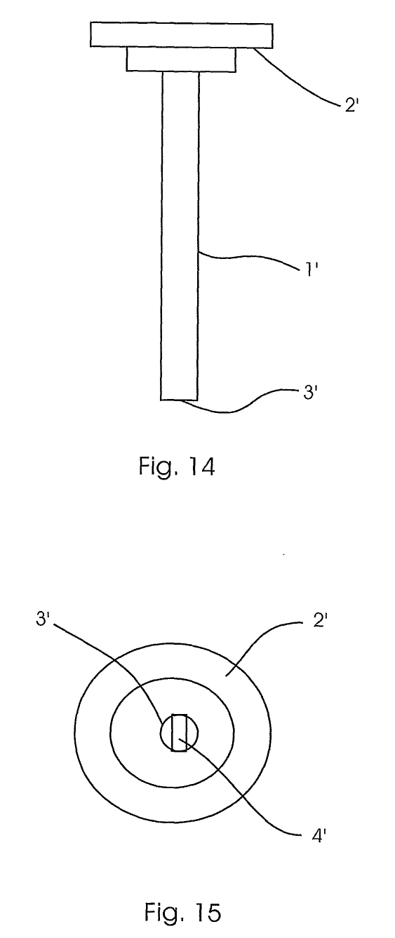

FIG. 14 shows a side view of a wand useful in a reagent transfer method;

FIG. 15 shows a bottom view of the wand of FIG. 14; and

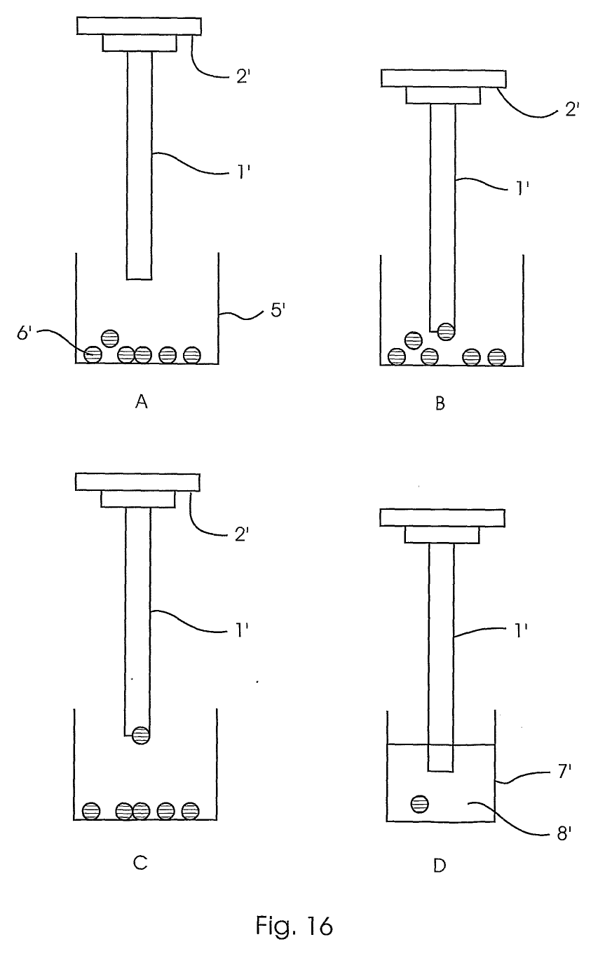

FIG. 16 illustrates schematically, a reagent transfer method.

FIG. 1 shows schematically, the operation of the method of the invention. The method is carried out in a reaction vessel 68 comprising an upper chamber 685 which has an open or openable mouth 686, and which opens into a lower chamber comprising a glass capillary tube 680. In this particular embodiment, the sides of the capillary tube 680 is coated with an electrically conducting polymer 681 and electrical contacts including a contact 683 at the lower end of the tube 680. A second contact (682) is provided around the base of the upper chamber (685) (FIG. 1B)

In first step, a first set of reagents, such as a PCR reaction mixture are dispensed into the upper chamber 685 of the reaction vessel. Surface tension will prevent these reagents entering the capillary tube 680. However, the reaction vessel 68 is pivotally mounted by spindles 72 on a platform or carousel, and subjected to a centrifugation step, as indicated by the curved arrows. This drives the PCR reaction mixture into the capillary tube as illustrated in FIG. 1(C).

At this point, one or more further reagents FIG. 1(D) are dispensed into the upper chamber 685. Again, surface tension will not allow them to enter the capillary tube 680. However, they are allowed to remain in the upper chamber 685 and the vessel 68 is closed by means of a cap 687 (FIG. 1E).

Thereafter, the reaction vessel is treated such that a reaction, such as a PCR reaction, takes place within the tube 680. In this particular embodiment, the reagents in the capillary tube 680 are thermally cycled by passing appropriate electrical current through the polymer 681 by way of contacts 682, 683. The further reagents are not able to take part in this reaction at this stage. Furthermore, the contact 682 acts as a heat sink to isolate them from the thermal cycles being conducted in the tube 680.

Once this reaction has been completed however, the further reagents can be added without removing the cap 687, by carrying out a second centrifugation step.

FIG. 2 shows perspective view of an apparatus 1 which is particularly suitable for carrying out the method of the invention. The apparatus comprises a platform 2 held in position by a twist lock 4. The platform comprises several chambers and functional components (illustrated also in FIG. 4). The platform rotates driven by a stepper motor 6 and a drive belt (not shown). The position of the platform is monitored using an index sensor (not shown) also by monitoring the movement of the stepper motor 6.

Located above the platform 2 is an arm 10 that comprises a fork 12 for removeably attaching to functional components (not detailed) on the platform 2. The arm 10 is shown in a raised position holding a vessel 68 above the platform 2. The apparatus also comprises a magnet 14 that is located directly above the fork of the arm 12. The magnet 14 is shown in the raised position.

The apparatus also comprises variety of devices and means which allow preliminary treatment of a sample, for instance a biological sample, to extract DNA therefrom. These include a heating means 16, which is also located above the platform 2 and shown in a raised position, and a means for sonicating a sample 18 again located above the platform 2 and shown in a raised position. The linear movement of the arm 10 and the magnet 14 is driven by a motor 20 attached to a drive belt 22 and controlled by a linear actuator 24. The linear movement of the heating means 16 and the means for sonicating a sample 18 is similar driven by motor 20 attached to drive belt 22 and individually controlled by linear actuators 26 and 28 respectfully. The apparatus also comprises a control panel 30 and a power source 32.

FIG. 3 shows a transverse cross section of the apparatus of FIG. 2. The components shown are the same as those shown in FIG. 2 except that linear actuator 24 cannot be seen from this view. This view additionally shows the drive belt 40 attached to motor 6 for rotating the platform and sensor 42 for sensing the position of the platform.

FIG. 4 shows a top view of the platform 2 of the apparatus which has been designed for processing a fluid sample prior to nucleic acid amplification. The platform is mounted on the apparatus using a twist lock mechanism 4. The platform comprises two functional components, a cutter 50 and a sheath 52. Each functional component comprises a lip 54 on either side that allows the functional component to interact with the arm of the apparatus (not shown). The lip is orientated such that as the platform rotates the forked component of the arm is able to slide under the lip of the functional component. The apparatus also comprises several chambers 56, 58, 60, 62, 64, and 66. Each of these chambers or reaction vessels has a different role, associated with the treatment of a biological sample such as a urine sample, to extract DNA from it to allow an amplification reaction to proceed. Chambers 56, 58, 60, 64, 66 are oval in cross section and comprise a circular well recess at the bottom of the chamber 560, 580, 600 and 640 respectively. Chamber 62 is circular in cross section. Any or all of the chambers may be covered with a metal laminate membrane prior to use, to keep reagents clean.

The apparatus further comprises a reaction vessel 68 which is a reaction vessel suitable for conducting a method of the present invention. This vessel has an upper chamber 685 which is circular in cross section, and which narrows to a lower chamber comprising a capillary tube at the base of the vessel indicated by 680. Reaction vessel 68 additionally comprises a lip 70 that allows the chamber to interact with the arm of the apparatus (not shown). Reaction vessel 68 is provided with spindles 72 which are pivotally mounted in sockets 74 on the platform 2. The reaction vessel 68 is also covered by a laminate metal membrane.

Suitably, as described above in relation to FIG. 1, the capillary tube 680 is coated with an electrically conducting polymer 681—(see FIG. 8), and has an upper electrical contact 682 just above the top end of the tube 680, and a lower electrical contact 683 at the lower end of the tube 680, disposed on the outside.

Chambers 60 and 62 are mounted together in a single container 76. This container 76 is detachable from the platform. The platform also comprises a cut away section 78.

The use of the apparatus and the platform for the processing of a fluid sample and then conducting a nucleic acid amplification in accordance with the method of the invention is set out below with reference to the above figures and additionally FIGS. 5 to 10.

A container 76 comprising sample chamber 60 is selected based on the chosen assay. Chamber 62 is preloaded with several reagents required for said assay. A fluid sample comprising a DNA analyte is collected and placed into the sample chamber 60. The sample chamber may be preloaded with a chaotropic salt that may lyse the sample such as guanidine hydrochloride, urea or sodium iodide. Magnetic binding beads 100 are then added to the sample and the lid of the sample container is closed. The container 76 comprising sample chamber 60, sample and reagent chamber 62 is loaded onto platform 2. Platform 2 is then loaded into the apparatus 1 and locked in place using twist lock 4. The arm 10 is lowered and the platform 2 rotated such that the fork 12 engages underneath the lip 52 of cutter 50. The arm 12 is then raised and the platform 2 then rotates such that chamber 56 is located under the cutter 50. The arm is lowered and cutter 50 pierces a laminated metal membrane (not shown) covering chamber 56. This is repeated such that the cutter 50 sequentially pierces the membranes covering chambers 58, 60, 62, 64 and 66 and reaction vessel 68.

FIG. 5 shows a cross section view of the operation of a functional component, here a cutter 50, piercing a laminated membrane (not shown) on to top of a chamber, for example 56, of the apparatus. The chamber 56 is attached to the platform 2. The figure illustrates the lip of the functional component 52 that is used to engage with the fork of the arm (not shown).

FIG. 6 shows a view to illustrate the detail of the attachment of a functional component, here a cutter 50, to the fork 12 of the arm 10. The fork 12 of the arm 10 engages with the cutter underneath the lip 52.

Once all of the laminated membranes of the apparatus have been pierced, the cutter 50 is returned to its original position on the platform 2 by rotation of the platform 2, lowering of the arm 10 and rotation of the platform in the opposite direction such that the fork of the arm 12 and the lip of the cutter 52 disengage.

The platform is then rotated such that the sample chamber 60 is now located underneath the means for sonicating the sample 18. The means for sonicating the sample 18 is lowered into the sample chamber 60 and the sonication of the sample is initiated. This provides a physical lysis step to lyse any spores that are present in the sample to release any DNA. At the same time the chaotropic reagent such as guanidine hydrochloride also acts to promote binding of DNA to the magnetic binding material to form a complex. When sonication is complete the means for sonicating the sample 18 is removed from the sample chamber 60. Prior to being stored the means for sonicating the sample 18 is first washed in two wash chambers, chambers 56 and 58. These chambers are preloaded with a suitable buffer, for example a 50% aqueous ethanolic solution 80. The means for sonicating the sample 18 is raised from the sample chamber 60, that platform 2 rotates such that buffer chamber 56 is now located underneath the means for sonicating the sample 18, the means for sonicating the sample lower into buffer chamber 56, activated briefly and raised. The procedure is repeated for chamber 58. After the second wash the means for sonicating the sample 18 is raised and stored.

The arm 10 is then lowered and the platform 2 is rotates such that the 12 engages underneath the lip 52 of sheath 54. The arm 10 is then raised thereby raising sheath 54 to above the platform 2. The platform 2 is then rotated such that the sample chamber 60 is directly underneath the sheath 54. The arm 10 is lowered thereby lowering the sheath 54 into the sample chamber 60. The magnet 14 is then lowered into sheath 54 and the magnetic beads 100 to which the DNA is bound are attracted to the sheath 54. The arm 10 is then raised thereby raising sheath 54 out of sample chamber 60. The magnet 14 is raised simultaneously with the arm 10 such that it remains inside the sheath 54.

FIG. 7 shows a cross section view of the operation of a functional component, here a sheath 54, with a magnet 14 to withdraw bound analyte from the sample chamber 60. The chamber 60 is attached to the platform 2. The sheath is lowered via the arm (not shown) into the sample 102 contained in the sample chamber 60. The magnet 14 is inserted into the sheath 54 and the magnetic beads 100 to which the DNA is complexed are attracted to the sheath 54.

The DNA bound to the magnetic beads 100 may then be washed, for example twice in a buffer such as an alcohol buffer, like a 50% alcohol buffer. The platform 2 is rotated such that first buffer chamber 64 containing an appropriate buffer solution is directly underneath the sheath 54 to which the magnetic beads 100 are attracted. The arm 10 is lowered, thereby lowering the sheath 54 into the buffer chamber 64. The magnet 14 is not lowered though. This means that the beads 100 are no longer attracted to the sheath 54 but instead detract and fall into the buffer solution. Rapid raising and lowering of the arm 10 and thereby sheath 54 in small vertical movements ensures that all beads 100 are released from the sheath 54 and are well mixed with the buffer solution. The sheath 54 is then lowered back into the first buffer chamber 64, the magnet 14 is lowered into the sheath 54 and the magnetic beads 100 with the DNA still bound reattach to the sheath 54. The process is repeated to wash the beads 100 in a second buffer comprising 50% aqueous ethanolic solution contained in a second buffer chamber 66. After washing the magnetic beads 100 with the DNA bound in the second buffer chamber 66 the arm 10 is raised thereby raising the sheath 54 and leaving the magnetic beads 100 in buffer chamber 66. The sheath 54 however is not returned to the platform 2 but instead is retained attached to arm 10.

The platform is now rotated such that the reaction vessel 68 is now directly underneath the means for heating 16. The reaction vessel 68 comprises a lower area 90 comprising a capillary tube 680 and an upper area 92. The lower area 90 is separated from the upper area 92 by an in tact laminated membrane 94. The upper area comprises a small volume, approximately 100 μl of water 96. The means for heating 16 is now lowered into the upper area 92 of reaction vessel 68 and is activated to heat the water 96 to a temperature of approximately 90° C. Once the water 96 is heated the means for heating 16 is raised and removed from the reaction vessel 68. The means for heating 16 is then stored on the apparatus 1 for future use.

FIG. 8 shows a cross section view of the operation of a physical processing means, here a means for heating 16, to heat a volume of solution 96, contained in an upper section of the upper chamber of the reaction vessel 68, of the apparatus 1. The reaction chamber is attached to the platform 2. The means for heating heats the water 96 that is held in the upper section 92 of the reaction vessel 68. The upper section 92 and the lower section 90 of the reaction vessel 68 are separated by an intact membrane 94.

The platform is again rotated such that the second buffer chamber 66 comprising the magnetic beads 100 to which the DNA remains bound is directly underneath the sheath 54. The arm 10 is lowered thereby lowering the sheath 54 into the second buffer chamber 66. The magnet 14 is again lowered into the sheath and again the beads 100 are attracted to the sheath 54. The sheath 54 and magnet 14 are both raised, that platform is rotated such that now the reaction vessel 68 is directly beneath the sheath 54. The arm 10 is lowered to lower the sheath 54 into the upper section 92 of the reaction vessel 68. As before, the magnet 14 is not lowered such that the beads 100 are no longer attracted to the sheath 54. The beads 100 are released into the upper section 92 of the reaction vessel 68. As previously small raising and lowering of the arm 10 and sheath 54 ensure that all beads are released from the sheath 54. The DNA is then eluted from the beads 100 by the warm water 96. The arm 10 is raised such that the sheath 54 is removed from the reaction vessel 68.

FIG. 9 shows a cross section view of the operation of a functional component, here a sheath 54, with a magnet 14 to release the bound analyte 100 into the reaction vessel 68. The magnetic beads 100 are released into the upper section 92 of the reaction vessel 68 where the heated water 96 elutes the DNA from the magnetic beads 100.

The platform is again rotated such that now the reagent chamber 62, into which have been pre-loaded the necessary reagents for a nucleic acid amplification reaction, is directly underneath the sheath 54. The arm 10 is lowered thereby lowering the sheath 54 into the reagent chamber 62. The reagents (not shown) have been pre formulated such that they are also bound to magnetic beads (not shown). Once the sheath 54 is in position in the reagent chamber 62 the magnet 14 is lowered into the sheath 54 and the magnetic beads to which the reagents are bound are attracted to the sheath 54. The sheath 54 and magnet 14 are together raised to remove the reagents (not shown) from the reagent chamber 62. The platform 2 is then rotated such that the reaction vessel 68 is now directly underneath the sheath 54. The arm 10 is then lowered thereby lowering the sheath 54 into the upper section of the reaction chamber 92. Again the magnet 14 is not lowered such that the magnetic beads to which the reagents are bound are released from the sheath 54 into the upper section 92 of the reaction vessel 68. The reagents are eluted from the magnetic beads by the warm water 96. After elution is complete the arm 10 is again lowered with sheath 54 in position. The magnet 14 is lowered into the sheath 54 and all of the magnetic beads in the upper section 92 of the reaction vessel 68, ie those from the analyte and for the reagent, are attracted to the sheath 54. The sheath 54 and magnet 14 are both raised to remove the beads and the platform 2 rotated. The beads are then deposited as waste into one of the used buffer chambers. After the beads have been released from sheath 54, the sheath is returned to its initial position on the platform 2 by again using movement of the arm 10 and rotation of the platform 2.

The upper section 92 of reaction vessel 68 now comprises a purified nucleic acid sample and all of the required reagents for an amplification reaction. The arm 10 is now used to pick up the cutter 50. The platform 2 again rotates such that the reaction vessel 68 is now directly underneath the cutter 50.

The arm 10 is lowered thereby lowering the cutter 50 into the reaction vessel 68. The cutter 50 pierces the membrane 94 and the water 96 containing the DNA and reagents drops into the lower section 90 of the reaction vessel 68. Rather than using the arm to remove the cutter 50, the cutter is instead left in position in reaction vessel 68 where it now acts as a stopper to seal the reaction vessel 68.

FIG. 9 shows a cross section view of the reaction vessel 68, here with a functional component, here the cutter 50, in position to seal the reaction vessel 68. The cutter 50 has also been used to pierce the membrane 94 separating the upper section 92 and the lower section 90 of the upper chamber in the reaction vessel 68 such that the water 96 containing the DNA analyte and the reagents for a nucleic amplification reaction can enter the capillary tube 680. The cutter 50 remains in place to seal the reaction vessel 68 such that no solvent can evaporate from the chamber during the amplification reaction. Alternatively a cap or other closure member can be applied to the reaction vessel 68.

In order to drive the water 96 containing the DNA and the reagents into the capillary tube 680 of the reaction vessel 68 the platform 2 is rotated at high speed. The centrifugal force drives the fluid 96 into the capillary tube 680. This is aided by the fact that, during rotation, the reaction vessel 68 is able to pivot on the platform by means of spindles 72 mounted in sockets 74. Furthermore in order to prevent the spillage of liquid contained in chambers 56, 58, 60, 64 and 66 during this high speed rotation, these chambers are designed with an oval cross section and a circular recess at the base, as shown in FIG. 3. This internal design prevents any spillage.

After the water 96 containing the DNA and the nucleic amplification has entered the capillary tube 680 the sample is ready to undergo a nucleic acid amplification reaction.

At this stage however, one or more further reagents are added to the section 90 of the reaction vessel 68. This can be done by first raising the cutter 50 using the arm 10, and then introducing the one or more further reagents. They may comprise further PCR reagents, required to do carry out a nested PCR reaction, or the reagents needed in a signalling system.

At this point the reaction vessel 68 can be capped, with a cap specifically for the purpose, or be introducing the sealing cutter 50 described above.

At this stage the reaction vessel 68 can be manually removed from the apparatus 1 for use in another apparatus where the nucleic acid amplification is conducted. In this instance however the single apparatus has been adapted to additionally conduct the nucleic acid amplification and optical detection thereof. These operations are performed in the lower half of the apparatus 1 (not shown).

In order to fully automate the process the reaction vessel 68 has been provided with a lip 98 such that it can be manipulated by the apparatus arm 10 in the same manner as other functional components 50 and 54.

The arm 10 is lowered and the platform 2 rotated such that the lip 98 of the reaction vessel 68 engages with the fork 12 of the arm 10. The arm 10 is then raised thereby raising the reaction vessel 68. The raised reaction vessel 68 is shown in FIGS. 1 and 2. The platform then rotates such that the cut away section 78 is now aligned underneath the raised reaction vessel 68. The arm is then lowered thereby lowering the reaction chamber through the cut away section 78 and into the lower part of the apparatus 1.

When located in the lower part of the apparatus 1 the nucleic acid amplification is performed using a thermal cycler to heat and cool the reaction mixture in the capillary tube 680 and an optical detector to detect the end products. This is aided by the fact that the capillary tube 680 is coated with an electrically conducting polymer 681, which allows rapid heating and cooling of the capillary tube 680. Specifically, the reaction vessel 68 can be placed into a socket of a controlled electrical supply such that the contacts 682 and 683 and the polymer 681 form a circuit. Control of the electricity supply to the circuit causes the polymer 681 to heat up or cool down, allowing a rapid thermal cycling.

Once this reaction has been completed, the reaction vessel 68 can be returned to the platform 2, to allow a second centrifugation step to be carried out, preferably using the arm 10. This will drive the further reagent or reagents into the capillary tube 680. The reaction vessel may then be subject to further processing as suits the particular further reagents added. For instance, it may be subjected to further thermal cycling, by a reiteration of the previous process, or a treated so that a signalling system is developed and/or detected.

For detection, the closed lower surface 684 of the capillary tube is preferably transparent, so that a visible signal can be read through it using a suitable detector device such as a spectrofluorimeter. This may be done by various means including transfer of the reaction vessel 68 to a spectrofluorimeter device, for instance using arm 10. Most preferably however, the spectrofluorimeter is arranged to read the signal from the reaction vessel 68 when it is in position in the socket of a controlled electrical supply used in the thermal cycling process.

After completion of the processing the platform 2 containing the cutter 50, the sheath 54 and chambers 56, 58, 60, 62, 64 and 66 and the reaction vessel 68 are all removed from the apparatus and disposed off. A new platform containing the necessary elements can then be introduced into the apparatus such that it can be used again in another sample manipulation.

FIG. 11A illustrates an arrangement whereby a reagent container (150) is positioned within the upper chamber (685) of a reaction vessel (68). This container (150) is provided with a plastic lid at its upper surface 154 and a foil membrane at its lower surface (152). Side walls of the container are compressible. The container is divided into individual compartments (153) (FIG. 11B) arranged annularly. Each compartment contains a different reagent.

The lid (154) (FIG. 11C) is provided with a plurality of piecing pins (156) arranged to pierce the lower (152) foil surfaces of each compartment (153) of the container (150). When pressure is application to the lid 154, for instance in step (3) of the method as described above, all the pins 156 pierce the lower surface (152) so that the reagents are delivered into the upper chamber (685).

If desired however, individual compartments can be pierced separately or sequentially, for instance by moving a piercing wand or cutter associated with the apparatus, so that it is aligned with the desired compartments individually, at the required time.

A modified form of such a container is illustrated in FIG. 12. In this case, the container (160) has a single chamber defined by upper and lower foil membranes (151, 152), and contains a liquid reagent (161). It is also provided with a pair of annular flanges (162, 163) at the upper region thereof, which define therebetween, a moulding (164) for a grabber arm (not shown).

This container can therefore be introduced automatically into the reaction vessel (68), or indeed into any other reaction vessel required. Piercing of the foils 151, 152 by means of a piercing pin on a lid, or a separate piercing wand, will release the contents of the chamber 161.

This container can therefore be used in the method described above, either to deliver reagents, for instance to the upper chamber (685) and then be removed from the vicinity. Alternatively, where it may be capped, for example with a lid containing a piecing pin, it may be left in-situ in the upper chamber (685) where it acts as a capping element.

An alternative multi-compartment container is illustrated in FIG. 13. In this case, the container (170) contains three reagents (171, 172, 173), the third of which contains magnetic beads (174), which are in separate compartments, defined by an upper foil membrane (151) and a lower foil membrane (152).

Piercing of the membranes (151) and (152) using a wand (175) will cause the reagents 171, 172 and 174 to be mixed sequentially together. The provision of magnetic beads allows for magnetic immunoseparation methods to be conducted.

FIG. 14 illustrates a wand which may be used, for example, to transfer reagents such as PCR ready beads into the container 686, for example in a preliminary step. Once in the container, the beads may be solubilised using for instance by addition of sample. Alternatively, the beads may be transferred into a different container for preliminary solubilisation and the resultant solution then transferred using, for instance a pipettor system.

The wand illustrated in FIG. 14 has an elongate shaft (1′) of an electrostatically charged or chargeable material such as polysytrene or latex. It is provided with a head structure (2′) which allows it to be attached to a device for automatically moving it from one position to another.

A lower surface (3′) of the shaft (1) is provided with a groove (4′) (FIG. 15) into which a reagent bead may be accommodated.

In use (FIG. 16), the wand is positioned in an automated assay device (not shown) by means of the head (2′). At least the lower region of the shaft (1) is electrostatically charged, for instance by having been rubbed against an insulating material. It is then positioned above a first container (5′) which holds reagent beads (6′) (FIG. 16A). The wand is lowered into the container (5′) until a bead (6′) is attracted to the charged surface (3′), where it becomes lodged in the groove (4′) (FIG. 16B).

The wand together with the attached bead (6′) can be raised out of the first container (5′) (FIG. 16C). A second container (7′) is then positioned under the wand. The second container contains a solvent or solution (8′). Lowering of the wand causes the surface (3′) to be immersed in the solution, whereupon, the bead is dispensed into the solution (FIG. 16D).

1. A method for carrying out a multi-step reaction, said method comprising
1) adding one or more first reagents to a reaction vessel, said reaction vessel comprising an upper chamber capable of holding reagents, which is open to a lower chamber to which reagent flow is restricted;
2) subjecting said reaction vessel to a centrifugal force so as to drive the said one or more first reagents into the lower chamber;
3) adding a further reagent to the first chamber and closing said chamber;
4) subjecting at least one of the lower chamber or the upper chamber to conditions which cause said one or more first reagents or said further reagent respectively, to take part in a first reaction or reach a desired reaction condition; and
5) subjecting said reaction vessel to a centrifugal force so as to drive the said further reagent into the lower chamber and allowing it to interact with contents of the lower chamber;
wherein at least steps (2) to (5) are carried out automatically.

2. A method according to claim 1 wherein the said lower chamber of the reaction vessel is a capillary tube.

3. A method according to claim 1, wherein during step 1, the lower chamber is subjected to conditions which cause said one or more first reagents to take part in a first reaction or reach a desired reaction condition.

4. A method according to claim 1 wherein the reaction vessel is closed during step (3) by means of an appropriately shaped lid.

5. A method according to claim 1 wherein the said one or more first reagents comprise a PCR reaction mixture, and during step (4) the reagents are subjected to thermal cycling.

6. A method according to claim 5 wherein thermal cycling is achieved by passing an electrical current through an electrically conducting polymer, which comprises or is contiguous with the upper or lower chamber.

7. A method according to claim 5 wherein the said further reagent comprises one or more further reagents required to carry out a second PCR reaction.

8. A method according to claim 5 wherein the said further reagent comprises a signalling system for detecting amplified nucleic acid.

9. A method according to claim 8 wherein the signalling system comprises reagents which will produce a fluorescent, chemiluminescent or bioluminescent signal in the presence of amplified DNA.

10. A method according to claim 9 wherein the signalling system is detectable, without opening the reaction vessel.

11. A method according to claim 9 wherein the signalling system comprises: a) a fluorescently labelled probe, which specifically binds a sequence found in DNA which would have been amplified during a PCR reaction, and b) a DNA binding agent, which is able to interact with the fluorescently labelled probe, by absorbing fluorescent energy emitted from the probe, or by donating fluorescent energy to the probe.

12. A method according to claim 11 wherein the DNA binding agent is one which does not emit fluorescent light when bound to DNA.

13. A method according to claim 1 wherein one of the said one or more first reagents or further reagent comprises the reagents necessary for carrying out a target reverse transcriptase process, and the other of said one or more first reagents or further reagent comprises the PCR reagents required to amplify a cDNA obtainable from said target reverse transcriptase process.

14. A method according to claim 5 wherein the said further reagent is a reagent able to degrade the products of the PCR amplification reaction.

15. A method according to claim 1 wherein said one or more first reagents comprise some of the reagents necessary for carrying out an amplification reaction, and wherein said further reagent comprises a reagent essential for said amplification reaction which is not contained within said one or more first reagents, and wherein, during step (4), the one or more first reagents is brought to a temperature condition which are favourable to the correct amplification occurring.

16. A method according to claim 1 wherein the one or more first reagents and/or the further reagent are provided in cartridges or breakable containers, disposed within the upper chamber above the opening into the lower chamber, prior to addition.

17. A method according to claim 16 wherein the one or more first reagents and/or the further reagent are added to the reaction vessel by breaking open the said cartridge or breakable container.

18. A method according to claim 17 wherein the said cartridge or breakable container is broken open using a piercing wand or cutter.

19. A method according to claim 1 wherein, in step 1), the one or more first reagents are solid reagents and these are transferred to the reaction vessel using a method comprising
(i) bringing into the vicinity of said solid reagents in a first container or first position a wand comprising an electrostatically charged material, said wand being capable of electrostatically attracting and retaining said solid reagents on the surface thereof, so as to pick up a quantity of said solid reagent;
(ii) moving the wand and/or the first container or reaction vessel so that the wand is in the vicinity of the reaction vessel; and
(iii) removing the solid reagent from the said wand, so that it is placed in the second container or second position.

20. A breakable container for storing reagents, said container having therein a reagent chamber with at least one pierceable wall at the lower surface thereof, and wherein the upper surface comprises either a further pierceable wall, or a lid comprising a piercing means, arranged such that piercing of said pierceable walls leads to release of reagent from the chamber.

21. A container according to claim 20 wherein the piercable walls are metal or laminated metal membrane surfaces.

22. A container according to claim 20 wherein the reagent chamber has more than one compartment.

23. A container according to claim 22 wherein the compartments are arranged adjacent each other.

24. A container according to claim 22 wherein the compartments are arranged on top of each other.

25. A container according to claim 20 which further comprises means to allow the container to be moved automatically into position within the reaction vessel.

26. A container according to claim 25 wherein said means comprises one or more annular flanges.

27. A method for transferring solid reagents from a first container or first position to a second container or second position, said method comprising:
(i) bringing into the vicinity of said solid reagents in the first container or first position a wand comprising an electrostatically charged material, said wand being capable of electrostatically attracting and retaining said solid reagent on the surface thereof, so as to pick up a quantity of said solid reagent;
(ii) moving the wand and/or the first or second containers so that the wand is in the vicinity of the second container or position; and
(iii) removing the solid reagent from the said wand, so that it is placed in the second container or second position.

28. A method according to claim 27 which is carried out automatically.

29. A method according to claim 27 wherein the solid reagent is a collection of reagents which has been freeze or spray dried.

30. A method according to claim 29 wherein the solid reagent is a bead comprising one or more reagents necessary for carrying out a nucleic acid amplification reaction.

31. A method according to claim 30 wherein the amplification reaction is a polymerase chain reaction.

32. A method according to claim 27 wherein the electrostatically charged material of the wand is polystyrene or latex.

33. A method according to claim 27 wherein, in a preliminary step, the charge on the wand is created or increased by rubbing the wand against an insulator.

34. A method according to claim 33 wherein the insulator is a synthetic fabric.

35. A method according to claim 33 wherein the rubbing step is carried out automatically.

36. A method according to claim 27 wherein the wand is hollow.

37. A method according to claim 36 wherein a magnet is provided and may be accommodated within the wand.

38. A method according to claim 27 wherein at least a part of the outer surface of the wand is profiled to allow reagents to be accommodated within the profiles.

39. A method according to claim 38 wherein the profiles comprise one or more dimples or grooves.

40. A method according to claim 38 wherein the lower surface of the wand is profiled.

41. A method according to claim 27 wherein in step (iii), the wand is immersed into a liquid to remove any solid reagent from the wand.

42. A method according to claim 41 wherein the solid reagent is a bead containing reagents suitable for carrying out a PCR reaction and the liquid is a resuspension buffer or DNA/RNA extract.

43. A method according to claim 27 wherein the wand is disposed after use.

44. An apparatus comprising a wand comprising a dielectric material which is electrostatically chargeable, and means for transferring said wand from a first container to a second container.

45. Apparatus according to claim 44 which comprises
(i) a platform comprising:
(a) a chamber suitable for receiving a sample; and
(b) a functional component;
(ii) an arm capable of being raised and lowered and including a means for removeably attaching to the functional component such that said component may be raised and lowered with the arm; and
(iii) a means for moving the platform such that any chamber or functional component may be aligned with respect to the arm,
wherein the said functional component is the wand.

46. A wand comprising a dielectric material which is electrostatically chargeable, and which is profiled so as to accommodate a solid reagent electrostatically in a groove or dimple on an outer surface thereof.
